(19) United States
(12) Patent Application Publication
(10) Pub. No.: US 2009/0196893 A1
(43) Pub. Date: Aug. 6, 2009

(54) Aqueous Cosmetic Composition, Especially for Use as Moisturizing Lotions

(75) Inventors: Jean-Francois Tranchant, Marigny Les Usages (FR); Thierry Pouget, Saint Cyr En Val (FR); Elodie Verdier, Mardie (FR)

Correspondence Address: HAMRE, SCHUMANN, MUELLER & LARSON, P.C., P.O. BOX 2902, MINNEAPOLIS, MN, 55402-0902, US

(73) Assignee: LVMH RECHERCHE, SAINT JEAN DE BRAYE (FR)

(21) Appl. No.: 12/227,249

(22) PCT Filed: May 15, 2007
(86) PCT No.: PCT/FR2007/051278

§ 371 Date: Feb. 5, 2009

(30) Foreign Application Priority Data
May 15, 2006 (FR) 0651742

(51) Int. Cl. A61K 8/34; A61Q 17/04; A61Q 19/02; A61K 8/02; A61Q 19/00

(52) U.S. Cl. 424/401; 514/772; 424/59; 424/62

(57) ABSTRACT

The invention relates to the use of an aqueous composition comprising at least one surface-active agent in the form of bilayers and exhibiting a structure of sponge phase type as cosmetic composition or for the preparation of a cosmetic composition.

It also relates to aqueous compositions, in particular lotions, in the sponge phase form including at least one cosmetic active agent or one cosmetic additive.

It also relates to a method for the cosmetic care of the skin or superficial body growths, in particular for the purpose of moisturizing them, and/or to a method intended to give fragrance to the skin or superficial body growths.

The invention relates to novel cosmetic compositions. More specifically, the invention relates to the use of structured aqueous phases of sponge phase type in the field of cosmetics and to novel cosmetic compositions in the form of aqueous phases of sponge phase type.

The present invention results from the discovery, by its inventors, that it is possible to prepare cosmetic compositions by formulating these compositions in the form of compositions of sponge phase type. Such cosmetic compositions have numerous advantages, as will be set out below.

The very specific structure named “sponge phase” structure has been described in several scientific publications of basic type. Mention will in particular be made of the publication by M Filali et al. in J. Phys. France (1994), 4, 349-365, entitled “L₃ (sponge) phase in the very dilute regime: spontaneous tearing of the membrane?”.

The structural study of the sponge phases has also formed the subject of the publication by Alibert C., et al., entitled “Dielectric study of the dilute part of a SDS/brine/alcohol system: a new sequence of phases?” in Europhysics Letters, 1997, 39(5), 563-568.

A person skilled in the art knows that a sponge phase is composed of a lyotropic system based on surfactants, in the dilute state.

European patent application EP 1 433 476 describes compositions intended for the cleaning of the skin which comprise an oil constituent (A), a hydrophilic nonionic surfactant (B), a lipophilic amphiphilic agent (C), a water-soluble solvent (D) and water (E).

The compositions described in this document are in the form of an isotropic liquid phase exhibiting a bicontinuous structure and exhibit, as a result of their composition, good detergency qualities and a good ability to be rinsed out.

However, to date, no cosmetic composition in the form of a sponge phase has been described.

It is specifically this novel cosmetic composition form which forms the subject matter of the present invention.

Specifically, the invention results from the observation by its inventors that the sponge phases intrinsically exhibit moisturizing properties with regard to the skin. Moreover, suitably formulated, they can result in cosmetic compositions and in particular in lotions exhibiting a particularly pleasant appearance and feel and, in addition, they provide a feeling of great freshness during and after the use thereof.

These compositions can in addition be formulated in order to exhibit various properties which are particularly advantageous in cosmetics, in particular various cosmetic properties related to the presence of cosmetic active agents specifically chosen for this purpose and/or of cosmetic additives.

It is also specified that the sponge phase of the invention has a particularly advantageous vectorizing effect with regard to the cosmetic active agent present therein, if appropriate.

Finally, the appearance of the compositions according to the invention, in particular when the latter are in the form of lotions, may be particularly attractive to the users owing to the fact that these compositions may be completely transparent and clear.

Thus, the present invention relates, according to a first aspect, to a novel use of sponge phases in the field of cosmetics.

According to a second aspect, the invention relates to novel cosmetic compositions in the form of sponge phases.

According to a third aspect, the invention relates to a process for preparing said compositions.

According to a fourth aspect, the invention relates to a cosmetic care method and/or a method intended to give fragrance to the skin or superficial body growths, in which a composition provided in the form of a sponge phase is applied topically to the skin or integuments.

A better understanding of the invention will be obtained in the light of the description and examples which follow, given with reference to the figures, which respectively represent:

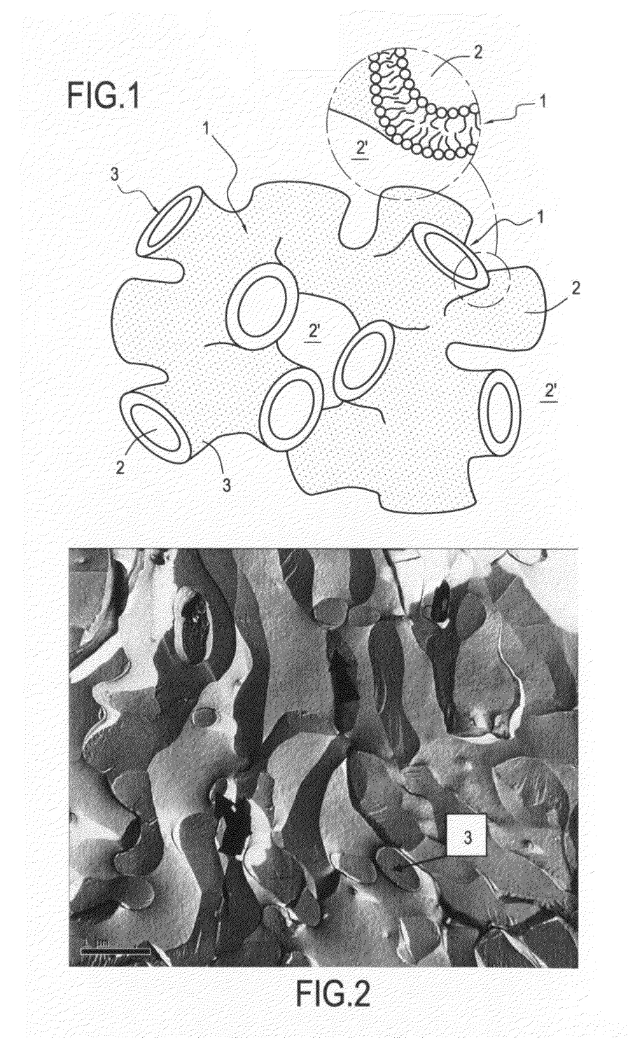

FIG. 1: the diagram of the structure of a sponge phase,

FIG. 2: an electron microscopy photograph of the freeze fracturing of a sponge phase, this photograph corresponding to the sponge phase obtained according to example 1,

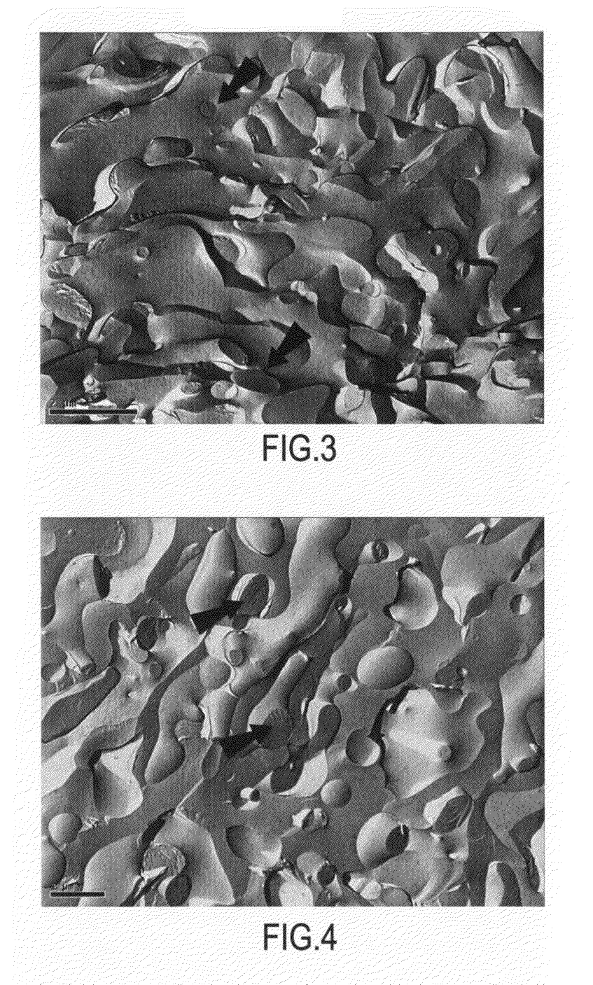

FIGS. 3 to 5: various photographs obtained by electron microscopy of the freeze fracturing of specific compositions according to the invention described in examples 2 to 4,

FIG. 6: an electron microscopy photograph of the freeze fracturing of the composition of example 2, after partial evaporation of the water.

A number of definitions of terms used in the present document are given below.

The term “sponge phases” denotes the phases as described in the various literature documents under this name and in particular in the documents mentioned at the start of the present document.

FIG. 1 diagrammatically represents such a sponge phase. Such a phase is composed of a lyotropic system in a dilute state and more specifically of aggregates structured in the dilute state in the form of an isotropic phase, formed of a single bilayer 1 folded over randomly in space and separating two equivalent solvent domains 2 and 2′. Such a structure is characterized by the presence of interconnected channels 3 of solvent (said solvent being water in the case of the present invention) which confer, on the combination, a structure reminiscent of that of a sponge, hence the name given to this type of phase.

This same FIG. 1 also gives a representation of an enlargement of the cross section of the channels diagrammatically showing the bilayer of surfactants 1 separating the two media 2 and 2′.

FIG. 2 gives a representation of the true form of a sponge phase, this phase being, in the present case, that obtained according to example 1, visible on an electron microscopy photograph of a freeze fracturing of such a phase. In this photograph, it is possible to distinguish the structure of this phase, with in particular the cross section of the channels 3 and the bilayer of surfactants 1 separating the two media 2 and 2′.

The definition of “cosmetic composition” is that which is given in the regulations in this field. The cosmetic composition is in particular a composition intended to be brought into contact with the various surface parts of the human body, in particular with the epidermis or with the integuments (hair and nails), for the purpose in particular of keeping them in good condition, of protecting them, of cleaning them, of giving fragrance to them or of modifying the appearance thereof.

In the context of the present invention, the cosmetic composition can in particular carry a cosmetically effective active agent. It can also in particular comprise a fragrancing agent or a fragrancing composition, whether it is a matter of giving fragrance to said cosmetic composition itself or whether this composition is intended to give fragrance to the skin or integuments.

The term “liquid composition” is understood to mean that the composition is in a fluid form capable of flowing. It can in particular exhibit a viscosity more or less in the vicinity of that of water. However, it is not forbidden, according to the invention, to introduce texturing agents into the compositions, for example viscosifying agents for adjusting the viscosity of the composition, provided, however, that the nature of these agents and their concentrations are chosen so as to adhere to the characteristic structure of sponge phase type of the composition.

An example of such a liquid composition is a cosmetic product, optionally comprising fragrance, used as a lotion for the care or hygiene of the skin, in particular of the epidermis, and/or of the hair.

The definition of “cosmetically effective active agents” is that conventionally known in the field of cosmetology. The agents involved are in particular the active agents used for the cosmetic care of the skin, such as moisturizing, cleaning, deep cleansing, tightening, toning, skin lightening, protective (in particular protective against ultraviolet radiation or other aggressive factors, such as the cold and atmospheric pollution), antiaging (in particular antiwrinkle and/or firming) and slimming cares.

Very clearly, some products, such as solvents, in particular glycerol, can have a cosmetic effect, as a result of their well known moisturizing properties, without, however, being regarded, within the meaning of the invention, as cosmetically active agents, insofar as such an agent actively contributes to the formation of the characteristic structure of the sponge phase, as will be set out below.

The definition of “cosmetic additive” relates, in the context of the present invention, to any ingredient used in cosmetic composition formulations for the purpose of conferring specific physical, chemical or sensorial properties thereon or to guarantee their stability, such as an antioxidant, a coloring agent, a fragrancing agent, a texturing agent or a preservative.

According to the essential characteristic of its first subject matter, the invention relates to the use of an aqueous composition comprising at least one surface-active agent in the form of bilayers and exhibiting a structure of sponge phase type as cosmetic composition or for the preparation of a cosmetic composition.

As indicated above, the actual structure of the sponge phases confers, on said aqueous compositions, particularly advantageous properties which led the inventors of the present invention to envisage their use in the field of cosmetics and very particularly as cosmetic composition. In the latter case, the aqueous composition itself, in the sponge phase form, constitutes the cosmetic composition of the invention.

In particular, by virtue of the intrinsic moisturizing properties of the structures of sponge phase type which are mentioned above, said cosmetic composition is moisturizing with regard to the skin.

A conductivity of 1 to 20 mS/cm of the composition provides the latter with its structure in the sponge phase form and with good stability. As emerges from the description which will follow, the invention provides various means of providing such conductivity, in particular by introducing satisfactorily conducting salts into the composition or more generally by varying the formulation and in particular by varying the nature of the surface-active agents present in the composition, it being understood, however, that the composition has to remain in the form of a lyotropic system in the dilute state.

In the context of the invention, it is possible in addition to introduce, into these compositions, one or more additional ingredients in order to confer thereon additional properties desired in the field of cosmetic compositions, care being taken that the choice of these additional ingredients and their proportions is made so that the characteristic structure of the sponge phase type is maintained.

Thus, the aqueous compositions, the use of which is targeted according to the present invention, exhibit a structure of sponge phase type and can be used according to the invention as cosmetic compositions.

Depending upon whether the cosmetic composition will or will not comprise such an additional ingredient, the sponge phase can be regarded either as the vehicle for such an additional ingredient or as the cosmetic product in itself.

Said additional ingredient will generally be composed of at least one cosmetic active agent or of at least one cosmetic additive.

The compositions used according to the invention, when they comprise such an additional ingredient composed of at least one cosmetic active agent and/or of at least one cosmetic additive, will constitute novel compositions in themselves and will form the subject matter of the second aspect of the invention.

Thus, the invention is targeted, according to its second subject matter, at a cosmetic composition comprising an aqueous composition in the sponge phase form, said sponge phase comprising at least one ingredient, referred to as additional ingredient, chosen from cosmetic active agents and cosmetic additives.

According to a preferred alternative form of this second subject matter, the cosmetic composition is composed in its entirety of said sponge phase including said additional ingredient.

The aqueous compositions used according to the invention and the novel compositions of the invention advantageously comprise at least one surface-active agent, at least one alcohol selected from the group consisting of glycol, glycerol, polyethylene glycols and C₄ to C₈ alcohols and optionally at least one alkali metal salt and/or at least one alkaline earth metal salt.

The novel aqueous compositions advantageously comprise at least one surface-active agent, at least one alcohol selected from the group consisting of glycol, glycerol, polyethylene glycols and C₄ to C₈ alcohols, at least one cosmetic active substance or at least one cosmetic additive and optionally at least one alkali metal salt or alkaline earth metal salt.

In order to simplify the account which follows, the term “compositions of the invention” will denote both the compositions used according to the invention and the compositions which constitute in themselves novel products.

A person skilled in the art knows that a sponge phase usually exhibits a conductivity of between 5 and 20 mS/cm. Thus, he will take care to control the conductivity during the preparation of the compositions of the invention.

The conductivity of the compositions of the invention is measured at 25° C. by methods usually known to a person skilled in the art. Thus, the conductivity of the composition of the invention, measured at 25° C., is between 1 and 20 mS/cm, preferably between 5 and 20 mS/cm.

It will be easily understood that the presence of a salt in the composition is not essential insofar as the formulation selected makes it possible to obtain the targeted conductivity without resorting to an additional salt.

The need to introduce a salt into the composition will depend on the nature of the cosmetic active agent or of the cosmetic additive, which may, for example, itself be in the salt form, and/or of the other constituents, in particular of the surfactant, which may contribute to obtaining the preferred conductivity of between 1 and 20 mS/cm, preferably of between 5 and 20 mS/cm.

In order to obtain the targeted conductivity, recourse will advantageously be made to an alkali metal salt or alkaline earth metal salt, preferably to an alkali metal salt, more preferably to sodium chloride.

The compositions of the invention advantageously comprise from 0.1 to 2% by weight of a C₄ to C₈ alcohol or of a mixture of C₄ to C₈ alcohols.

Hexanol will be chosen as preferred alcohol.

The compositions advantageously comprise glycerol and/or glycol. Glycol or glycerol will advantageously be present in the composition at a concentration by weight of between 0.1 and 50%, preferably between 20 and 40%.

Glycerol is very particularly advantageous in obtaining the cosmetic compositions of the invention insofar as it furthermore confers on these compositions a particularly moisturizing nature.

In order to obtain a sponge phase, it is necessary for the composition to comprise at least one surface-active agent.

These surface-active agents can be chosen from anionic, cationic, amphoteric and nonionic surfactants and their mixtures. These various surface-active agents are well known to a person skilled in the art. Reference may be made, among the many works dealing with these compounds, to the “Handbook of Surfactants” by M. R. Porter, 1991, published by Blackie (Glasgow).

The surface-active agent or the mixture of surface-active agents will generally be present in the aqueous compositions of the invention at concentrations of between 0.2 and 2% by weight, with respect to the whole of the composition.

Mention will be made, as example of surfactants which can be used according to the invention, of sodium dodecyl sulfate, calcium dodecyl sulfate, cetylpyridinium chloride, ethoxylated stearyl alcohols (Steareth-100), surfactants of amine oxide type or dodecyltrimethyl-ammonium bromide (DTAB).

The choice will preferably be made to resort to at least one ionic and preferably anionic surface-active agent, in particular sodium dodecyl sulfate or calcium dodecyl sulfate.

Even if one of the advantages of the compositions of the invention is that they exhibit a particularly pleasant appearance and feel as a result of their characteristic structure, while having a very low viscosity comparable with that of water, it is not out of the question to introduce, into these compositions, an agent which makes it possible to adjust their viscosity, such as a texturing, thickening or gelling agent. Mention will be made, as example of such viscosifying agents, of alginates, chitosan and its derivatives, bacterial polysaccharides, such as Alcaligenes polysaccharides, and hydroxyethylcellulose.

This viscosifying agent will then be chosen, and also its concentration, so as to adhere to the characteristic structure of the sponge phases.

Finally, a person skilled in the art will easily understand that, provided that the formulation of the whole of the composition is varied, it is possible to incorporate therein various additional ingredients, such as active agents of advantage in the field of cosmetics or cosmetic additives intended in particular to render the compositions more attractive to the users thereof.

Such additional ingredients will generally be present in the compositions of the invention at concentrations of less than 10% by total weight, with respect to the weight of the composition. A person skilled in the art will easily understand that, for each type of additional ingredient, the lower concentrations will generally be those conventionally used in the field.

Of course, systematic trials will be used to choose both the concentration and the nature of these various additional ingredients so as not to harm the stability of the sponge phase.

According to a first alternative form, the composition of the invention will comprise at least one cosmetically active agent.

Such an agent will advantageously be selected from the group consisting of moisturizing, cleaning, deep cleansing, tightening, toning, skin lightening, protective (in particular protective against ultraviolet radiation or other aggressive factors, such as the cold and atmospheric pollution), antiaging (in particular antiwrinkle and/or firming) and reducing agents, skin lightening agents, agents having an antiaging activity with regard to the skin, skin protective agents, in particular agents having an activity against free radicals, and vitamins.

Mention will be made, as example of such cosmetic active agents, as moisturizing agents, of ectoine and sea water, as skin lightening agents, of kojic acid and ascorbic acid and their esters, as protective agents, of sunscreens, as antiaging agents, of agents which scavenge free radicals, peptides, vitamin A and its esters.

The choice will preferably be made, among cosmetically active agents, of those which are soluble in water, for reasons of simplification of the formulation.

A person skilled in the art will easily understand that it is also possible to choose additional ingredients which would not be soluble in water, provided that they are suitably formulated so as to allow them to dissolve in the aqueous phase used for the preparation of the sponge phase and that they are used in proportions compatible with the production of such a sponge phase.

A particularly advantageous cosmetic active agent which can be introduced into the sponge phases of the invention is ectoine, which is a natural molecule which makes it possible to maintain the equilibrium of the osmotic pressure in the cell and to stabilize biopolymers (proteins, nucleic acids, membranes) forming a protective hydric shield with regard to attacks (heat, dehydration). This specific active agent is advantageous through its gelling and moisturizing properties.

Ectoine can be present in the compositions of the invention at concentrations of between 0.01 and 10% by weight.

According to another alternative form, the composition of the invention will comprise, as additional ingredients, at least one fragrancing agent which confers a fragrant nature on said composition.

As in the case of the above cosmetic active agents, the fragrancing agent will advantageously be a water-soluble fragrancing composition or a fragrancing composition formulated in order to be soluble in water.

According to yet another alternative form, the composition of the invention can comprise, as additional ingredient, a dye.

It will preferably be a water-soluble dye. Mention will be made, as example of such dyes, of those referenced FDC Blue 1 and FDC Yellow 5.

As set out above, the presence of a salt is not always essential from the moment when the desired conductivity is obtained via other constituents of the composition.

When, in order to obtain this conductivity, recourse will be had to the presence of at least one salt, this or these salts will advantageously be present at a content of between 0.1 and 5% by weight, with respect to the weight of the composition.

The choice will advantageously be made to use sodium chloride as salt intended to introduce the conductivity targeted in order to obtain the sponge phase.

Finally, the composition can comprise a preservative, for example a cosmetically acceptable ester or mixture of esters of para-hydroxybenzoic acid (of paraben type), generally at a concentration of between 0.001 and 0.3% by weight, with respect to the total weight of the composition.

The composition will advantageously be prepared in order to be compatible with topical application to the skin or integuments and will advantageously be formulated in the form of a lotion.

The various ingredients will preferably be chosen for their compatibility with such an application.

The invention also relates to the process for the preparation of the novel compositions of the invention.

Such a process comprises the mixing of at least one surface-active agent with at least one alcohol chosen from glycol, glycerol, polyethylene glycols and C₄ to C₈ alcohols, at least one active substance and optionally at least one salt, in proportions such that said composition exists in the form of a sponge phase.

In an advantageous implementation of the process of the invention, the various constituents of the composition of the invention are incorporated in proportions such that said sponge phase exhibits a conductivity, measured at 25° C., of between 1 and 20 mS/cm, preferably of between 5 and 20 mS/cm.

As described above, the surface-active agent or the mixture of surface-active agents can be chosen from anionic, cationic, amphoteric and nonionic surfactants and their mixtures.

Preferably, an ionic surface-active agent and more preferably an anionic surface-active agent will be chosen.

According to an advantageous alternative form of this process, an aqueous brine, in particular a brine based on water, on glycerol or on glycol and on sodium chloride, will first of all be prepared, in which the other ingredients of the composition will subsequently be incorporated, the proportions of the various ingredients being defined so as to obtain a sponge phase.

Such an aqueous brine will advantageously comprise from 20 to 50 parts by weight, preferably from 25 to 45 parts by weight, of glycerol and from 0.1 to 3 parts by weight, preferably from 0.5 to 2.5 parts by weight, of sodium chloride, the remainder of this brine being composed of water.

The other constituents of the composition will subsequently be introduced into this brine, so as to obtain a sponge phase.

Such a choice of a composition particularly rich in glycerol, such as that obtained in the process described above, exhibits the advantage of easily resulting in compositions exhibiting a sponge phase structure but also results in compositions which exhibit all the advantages inherent in a composition rich in glycerol, namely a moisturizing nature, gloss properties related to a high refractive index and a good ability to render the skin more malleable and softer.

Finally, according to its final aspect, the invention relates to a cosmetic care method and/or a method intended to give fragrance to the skin or integuments comprising the topical application, to the skin or integuments, of an aqueous liquid composition as defined above or as obtained according to the process defined above.

In particular, the abovementioned method makes it possible to improve the moisturizing of the skin.

This is because, as set out above, the compositions of the invention are particularly advantageous as a result of their texture and as a result of their moisturizing properties related to the presence of the water channels but also related to the nature of the active agents and of the solvents which may be present in the composition.

In particular, use will be made of sponge phases which are rich in glycerol, it being possible for this content to range up to 50% by weight, with respect to the weight of the sponge phase, in order to obtain particularly valued moisturizing properties, in particular for the preparation of compositions of shittori type very particularly prized on the Asian market.

The compositions used in the method defined above additionally exhibit the advantage of resulting, during application to the skin and by evaporation of a portion of the solvent, in a true encapsulation of the active agent in the structure formed by the surface-active agent.

This is because it is apparent that, during the evaporation, in particular during the evaporation which takes place on contact of the product with the skin, the system becomes concentrated and the sponge phase is converted to a vesicular phase, the vesicles being formed by the lamellar structure of the surface-active agent.

It will be specified that the aqueous compositions in the sponge phase form, in particular in the form of lotions, are very clear, which is not necessarily the case with an aqueous suspension of vesicles formed of bilayers of surfactants.

The following examples are given without implied limitation of the invention and are illustrated by FIGS. 2 to 6.

### EXAMPLES

In all the following examples, the proportions are given as percentage by weight.

### Example 1

### Moisturizing Lotion in the Sponge Phase Form

A brine is prepared by mixing:
sodium chloride: 1%,
glycerol: 30%,
water: 69%.

1.5 parts by weight of hexanol, 0.6 part by weight of sodium dodecyl sulfate and 0.01 part by weight of methylparaben, as preservative, are added to 97.6 parts by weight of this brine.

After stirring the mixture obtained for 10 to 15 min, a transparent composition is obtained which is particularly soft to the touch and which exhibits a moisturizing nature with regard to the skin.

FIG. 2 is a photograph obtained by freeze fracturing which clearly shows the structure of sponge phase type of the composition obtained.

The conductivity of the moisturizing lotion is measured at a temperature of 25° C. using a Tacussel CD810 conductivity meter and a Tacussel platinum electrode (5×5 mm, crystal XM 200), a standard solution being employed.

The conductivity measured for the moisturizing lotion prepared above is 5.25 mS/cm.

### Example 2

### Moisturizing Lotion in the Sponge Phase Form Comprising Ectoine

A brine based on water, on glycerol and on sodium chloride is prepared.

This brine comprises 0.5% by weight of sodium chloride and 30% by weight of glycerol.

Starting from this brine, a mixture is prepared in the following proportions by weight:
ectoine: 0.2%
sodium dodecyl sulfate: 0.6%
hexanol: 1.5%
brine: 97.7%

After stirring for 10 to 15 min, the photograph represented in FIG. 3 is observed by freeze fracturing.

In this figure, the arrows indicate the presence of channels of solvents.

The transparent composition obtained exhibits a particularly pleasant feel. It also exhibits a high moisturizing power with regard to the epidermis.

The conductivity of the moisturizing lotion is measured at 25° C. using the equipment described in example 1.

The conductivity measured for the composition of example 2 is 2.6 mS/cm.

### Example 3

### Fragrant Moisturizing Lotion in the Sponge Phase Form Comprising a Fragrancing Composition

Starting from the brine prepared in example 2, a composition is prepared which comprises:
0.3% by weight of a commercial fragrance concentrate (sold by Takasago Europe Perfumery Laboratory, France, under the designation TPF 1433)
0.6% by weight of sodium dodecyl sulfate and
2% of hexanol.

The freeze fracturing photograph obtained from the composition prepared above is represented in FIG. 4 and the arrows indicate the presence of the channels characteristic of the sponge phase.

The transparent composition obtained exhibits a particularly pleasant feel and a particularly pleasant fragrance.

### Example 4

### Moisturizing Lotion in the Sponge Phase Form Comprising a Coloring Agent

Starting from the brine prepared in example 1, a composition is prepared which comprises:
0.1% by weight of an aqueous solution of the blue dye FDC Blue 1,
0.6% by weight of sodium dodecyl sulfate,
1.5% by weight of hexanol,
98.2% by weight of the brine.

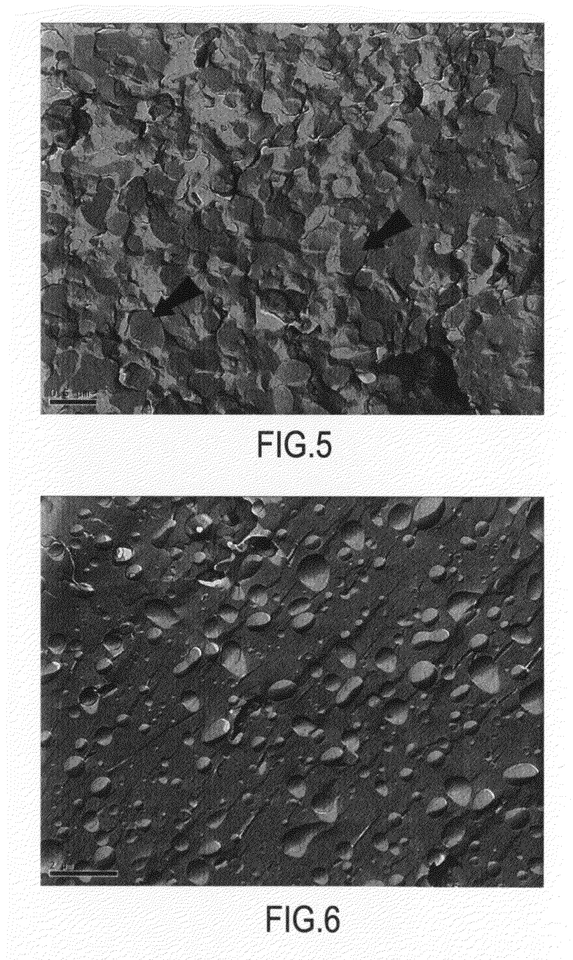

As is apparent in FIG. 5, the composition thus obtained, examined by freeze fracturing, exhibits the structure characteristic of a sponge phase, the channels characteristic of this structure being indicated by arrows.

The transparent composition obtained not only exhibits a particularly pleasant feel but is colored blue.

### Example 5

### Demonstration of the Formation of the Vesicles by Evaporation

It was observed by the inventors that the aqueous compositions according to the invention spontaneously formed vesicles after the evaporation, even partial evaporation, of the water present in the sponge phase.

Thus, when these compositions are in particular applied to the skin or to the integuments, in the context of a cosmetic use according to the invention, vesicles are formed on the applicational surfaces as the water present therein evaporates. The wall of these vesicles is formed of one or more bilayers of the surfactant which structured the initial sponge phase.

In the case in particular where these compositions comprise cosmetic active principles, the latter are thus vectorized by these vesicles to their sites of action.

In order to demonstrate the formation of these vesicles, the moisturizing lotion of example 2 is evaporated by gentle heating under reduced pressure down to a water loss of 10%. Freeze fracturing of the composition thus obtained, observed by electron microscopy, is represented in FIG. 6. It is possible to distinguish therein a large number of vesicles which were formed from the initial sponge phase corresponding to the freeze fracturing photograph represented in FIG. 3.

1. A method of use of an aqueous composition comprising at least one surface-active agent in the form of bilayers and exhibiting a structure of sponge phase type as a cosmetic composition or for the preparation of a cosmetic composition.

2. The method as claimed in claim 1, wherein the aqueous composition is used as a cosmetic composition.

3. The method as claimed in claim 1, wherein said cosmetic composition is a moisturizing composition for the skin.

4. The method as claimed in claim 1, wherein said aqueous composition comprises at least one surface-active agent, at least one alcohol selected from the group consisting of glycol, glycerol, polyethylene glycols and C₄ to C₈ alcohols and optionally at least one alkali metal salt or alkaline earth metal salt.

5. The method as claimed in claim 1, wherein said aqueous composition exhibits a conductivity, measured at 25° C., of between 2 and 20 mS/cm.

6. The method as claimed in claim 1, wherein said aqueous composition comprises from 0.1 to 2% by weight of a C₄ to C₈ alcohol or of a mixture of C₄ to C₈ alcohols.

7. The method as claimed in claim 1, wherein said aqueous composition comprises from 0.1 to 50% by weight of glycerol and/or glycol.

8. The method as claimed in claim 1, wherein said aqueous composition comprises from 0.2 to 2% by weight of at least one surface-active agent selected from the group consisting of anionic, cationic, amphoteric and nonionic surface-active agents and their mixtures.

9. The method as claimed in claim 8, wherein said aqueous composition comprises at least one ionic surface-active agent.

10. The method as claimed in claim 1, wherein said aqueous composition comprises from 0.1 to 5% by weight of at least one alkali metal salt or alkaline earth metal salt.

11. The method as claimed in claim 1, wherein said aqueous composition additionally comprises an ingredient, referred to as additional ingredient, selected from the group consisting of:
cosmetic active agents, in particular agents selected from the group consisting of moisturizing, cleaning, deep cleansing, tightening, toning, protective against ultraviolet radiation or other aggressive factors, and slimming active agents, and
cosmetic additives selected from the group consisting of cosmetic active agents.

12. The method as claimed in claim 1, wherein said cosmetic composition is formulated in the form of a lotion.

13. An aqueous cosmetic composition, which comprises an aqueous composition in the sponge phase form comprising at least one surface-active agent in the form of bilayers and exhibiting a structure of sponge phase type as a cosmetic composition or for the preparation of a cosmetic composition, said sponge phase comprising at least one ingredient, referred to as additional ingredient, chosen from cosmetic active agents and cosmetic additives.

14. (canceled)

15. The composition as claimed in claim 13, which exhibits a conductivity, measured at 25° C., of between 1 and 20 mS/cm.

16. The composition as claimed in claim 13, which comprises at least one cosmetic active agent selected from the group consisting of moisturizing, cleaning, deep cleansing, tightening, toning, protective against ultraviolet radiation or other aggressive factors, and slimming active agents.

17. The composition as claimed in claim 16, which comprises at least one moisturizing agent and/or at least one skin lightening agent and/or at least one protective agent.

18. The composition as claimed in claim 16, which comprises ectoine.

19. The composition as claimed in claim 13, which comprises a cosmetic additive selected from the group consisting of antioxidants, coloring agents, fragrancing agents, texturing agents and preservatives.

20. The composition as claimed in claim 19, which comprises a dye.

21. The composition as claimed in claim 19, which comprises a fragrance or a fragrancing composition.

22. The composition as claimed in claim 19, which comprises at least one texturing agent which makes it possible to adjust its viscosity.

23. The composition as claimed in claim 19, which comprises at least one preservative.

24. The composition as claimed in claim 13, which comprises from 0 (excluded) to 10% by weight of said additional ingredient(s).

25. The composition as claimed in claim 13, wherein said additional ingredient or at least one of said additional ingredients is a water-soluble compound or a compound formulated so as to be soluble in water.

26. The composition as claimed in claim 13, which comprises from 0.1 to 5% by weight of at least one alkali metal salt or alkaline earth metal salt.

27. The composition as claimed in claim 13, which is provided in the form of a lotion.

28. A process for the preparation of an aqueous composition as claimed in claim 13, which comprises the mixing of at least one surface-active agent, at least one alcohol selected from the group consisting of glycol, glycerol, polyethylene glycols and C₄ to C₈ alcohols, at least one active substance and optionally at least one salt, in proportions such that said composition exists in the form of a sponge phase.

29. The process as claimed in claim 28, wherein the preparation is carried out of an aqueous brine comprising, per hundred parts by weight, from 20 to 50 parts, of glycerol and from 0.1 to 3 parts, of sodium chloride, the remainder of said aqueous brine being composed of water, and incorporation is carried out, in said brine, of the other components of said cosmetic composition, so as to obtain a sponge phase.

30. The process as claimed in claim 28, wherein the constituents of said cosmetic composition are incorporated in proportions such that said sponge phase exhibits a conductivity, measured at 25° C., of between 1 and 20 mS/cm.

31. A method for the cosmetic care of the skin or integuments and/or a method intended to give fragrance to the skin or integuments, which comprises the topical application to the skin or integuments in need thereof of an aqueous liquid composition as defined in claim 13.

32. The method as claimed in claim 31, which is intended to improve the moisturizing of the skin.
